(19) United States
(12) Patent Application Publication
(10) Pub. No.: US 2005/0055736 A1
(43) Pub. Date: Mar. 10, 2005

(54) Method and Apparatus for Separating Oil Seeds

(76) Inventors: Charles Hepfner, Morinville (CA); Scott Hepfner, Morinville (CA); Neal Hepfner, Morinville (CA); Dale Hepfner, Morinville (CA)

Correspondence Address: Charles Hepfner, 9501-103 ST, Morinville, T8R 1G1, CA

(21) Appl. No.: 10/711,174

(22) Filed: Aug. 30, 2004

Related U.S. Application Data
(60) Provisional application No. 60/500,808, filed on Sep. 4, 2003.

(51) Int. Cl. C12N 15/00

(52) U.S. Cl. 800/23

(57) ABSTRACT

The invention relates to a method and apparatus for separating oil seeds, for example canola seeds, on the basis of ripeness. Components of a seed mixture are fed onto a flat plate such that the seeds are allowed to roll generally independently. The flat plate is positioned to decline downwardly from the feed end to the discharge end so as to allow the components to be separated by rolling at different rates according to the ripeness of the seeds. A collection means at the discharge end of the flat plate is adapted for selectively collecting, at one or more timed intervals, the components of the seed mixture such that ripe seeds are separated as the earliest to reach the discharge end of the plate.

### BACKGROUND OF THE INVENTION

Developed from rapeseed, through plant breeding and research, canola is an oil seed crop grown mainly in parts of western Canada, with some acreage in Ontario and the Pacific northwest, north-central and south-eastern United States. Its yellow flowers produce pea shaped pods that contain tiny seeds harvested for their oil. Canola is graded for several physical characteristics including the ratio of green canola seeds to yellow canola seeds and the ratio of damaged canola seeds to yellow canola seeds. The relative green seed count and damaged seed count are two of the primary determinants for the grade and hence the value of canola. Yellow canola seeds are ripe canola seeds whereas green canola seeds are mainly unripe seeds. The higher the amount of green and damaged canola seeds in relation to yellow canola seeds, the lower the grade of canola, and hence the lower the commercial value for the crop. The problem in differentiating between the two types of seeds, however, is that all canola seeds have a black shell, making them visually identical in their unprocessed form and only after crushing can one determine the grade and ripeness of a given sample. Unripe seeds are distinctly green when crushed, whereas ripe seeds are yellow. Based on crushed seed examination, No. 1 and No. 2 Canada canola grades may contain a maximum of 2.0% and 6.0% distinctly green seeds, respectively. Seed crushers remove the green from the oil with bleaching clays, which produce an added expense, absorb a fair bit of the oil in the process and pose an environmental problem. Additionally, unripe seeds are green because of their chlorophyll content. The relationship between high chlorophyll content and reduced seed vigor is well documented. By removing unripe seeds the overall vigor of a canola seed mixture can be improved. There remains absent in the current art an apparatus or method for separating unripe seeds from ripe seeds, and thereby significantly improving both the grade and vigor of canola seed.

There are 4 methods commonly used in the agriculture industry for removing unwanted material from desired seed. These include the following:
1. Mills: Seed is passed over a screen with holes of a specific size allowing seed to fall through, while retaining the chaff (overs) or by retaining the seed and allowing smaller debris to fall through (thrus). This method separates the wanted from the unwanted material on the basis of size.
2. Gravity Tables: Seed is passed over a sloped vibrating table against a current of air causing light material to move to one side, and heavier material to move to the other. Separation is based on the specific gravity differences between the wanted and unwanted material.
3. Aspirators: Air is passed through agitated seed, drawing away light chaff and debris. This method uses the differences in weight between the seed and the light chaff and debris as separation criteria.
4. Indent machines: Seed is passed within a pitted roller with indentations whose dimensions approximate the size of the seed to remove those materials whose size does not match.

Much of the prior art in considering the problem of separating the wanted from the unwanted in grading and cleaning seeds have employed similar methods to the ones listed above. For example U.S. Pat. No. 4,280,900 issued Jul. 28, 1981, to Gjesdal teaches a seed cleaner (separator) which separates all materials thicker than the desired seed, passes the remaining fraction through an air cleaner to remove chaff, dust and light fractions and then separates the remainder by screening out seed and the like which is thinner than the desired fraction. U.K. Patent Application No. GB 2,213,079 A published Aug. 9, 1989, to Drury teaches projecting grains or seeds at the same velocity in a stream with a rotary impeller wherein material of different specific gravity will be caught at different catching sites and collected separately. Given the similarities in the weight, size and shape between yellow and green canola seeds, a much more sensitive approach to separation must be considered.

There are a number of machines in the prior art, which utilize rolling as a method for separating material, however, many of these do not apply to grain and oil seeds. Those that do apply could not be used for seeds such as canola or mustard because of the degree of refinement required to separate ripe and unripe seeds. Additionally, attempts at a rolling method in the prior art suffer from insufficient output.

For example Canadian Patent No. 206,081 issued Nov. 30, 1920, to Richardson, discloses a grain separator wherein the seeds run onto a sloped conveyor belt with wire mesh netting. With this device, all of the granular material having a sufficiently smooth external surface to pass over the wires of the netting will readily slip down to the lower edge of the belt. Meanwhile the rougher, and smaller pieces of grain or seeds will be held more nearly to their original relation to the sides of the belt when received upon and be carried longitudinally thereof to and over the discharge roller at the end of the belt. This device lacks the sensitivity to separate seeds which are physically very similar, like green and yellow seeds of canola. U.S. Pat. No. 1,291,278 issued Jan. 14, 1919, to Ulrich, separates seed by feeding onto a rolling inclined plane. The slope of the plane is such that wheat or other grain having irregular surfaces will remain on the plane, while the spherical vetch or other seeds roll off the lower edge. Again, since oil seeds, such as yellow and green canola seeds, share similar size, weight and shape, this method would fail to maximize the differential rolling of ripe versus unripe oil seeds. Green seeds and yellow seeds roll differentially as a characteristic of their ripeness. Also, the seeds of canola or mustard would be unable to move unrestricted along the length of a plane under this method. A problem with a differential rolling method for separating oil seeds is how to feed the seeds onto the separating surface in such a manner that allows each oil seed to move unrestricted along its length. In every one of the preferred embodiments described in the prior art, there would be an inability for the oil seeds, for example canola seeds, to separate efficiently. The prior art approach lacks the ability to feed seeds onto a flat plate as a generally single layer of seeds to allow the seeds to roll generally independently.

U.S. Pat. No. 4,301,931 issued Nov. 24, 1981, to Satake, sorts grain by feeding on an inclined sorting board but has a surface which is far too course for separating oil seeds such as canola or mustard seeds. U.S. Pat. No. 1,466,560 issued Apr. 28, 1923, to Raze, teaches the use of frictional resistance down an inclined sheet to clean and separate seeds. With this method those seed offering a greater degree of frictional resistance will remain on the platform while those seed offering the least resistance, such as round seed, will roll forwardly and off the end of the platform. With canola, as with mustard, both green and yellow seeds are substantially round and it is a characteristic of their relative smoothness, rather than their shape alone, that determines their rate of rolling. The prior art lacks the sensitivity to separate oil seeds on the criteria of their ripeness.

There remains to be found in the prior art an effective method and apparatus for separation and grading of oil seeds. Many oil seeds, such as canola seeds, cannot be separated on the basis of ripeness using size, weight, color or shape alone as indicators. There is a need for an apparatus and method for separating oil seeds on the basis of ripeness.

### SUMMARY OF THE INVENTION

In searching for ways to improve the quality of canola crops, the inventors made a surprising discovery on the rolling characteristic of yellow and green seeds of canola. On rolling canola seeds on a slanted piece of glass to remove chaff from a sample, the inventors discovered, surprisingly that if only part of the canola seeds were rolled to the base, the seeds at the base tended to have a better ripe count than the seeds which remained closer to the top of the glass. They discovered that canola seeds can be separated on the basis of differential rolling characteristics and that yellow seeds tend to have a lower differential rolling resistance than counterpart green seeds. Green seeds and otherwise damaged seeds tend to have a slightly rougher, more wrinkled surface than yellow seeds, which are generally fuller and rounder. To the inventors' knowledge, this significant relationship had remained undetected to date. Using this differential characteristic, the inventors created a novel apparatus and method for seed separation of oil seeds.

In one broad aspect, the present invention provides an apparatus for separating components of a seed mixture of ripe seeds, unripe seeds, and debris, said apparatus comprising:
a frame;
a hopper mounted on the frame for holding components of the mixture;
a flat plate connected to the frame with a proximal feed end and a distal discharge end, wherein the flat plate is located beneath the hopper and is positioned to decline downwardly from the feed end to the discharge end so as to allow the components to be separated by rolling at different rates according to the ripeness of the seeds;
feeding means carried by the frame for feeding the components from the hopper onto the feed end of the flat plate as a generally single layer of seeds to allow the seeds to roll generally independently;
collection means at the discharge end of the flat plate adapted for selectively collecting, at one or more timed intervals, the components of the seed mixture such that ripe seeds are separated as the earliest to reach the discharge end of the plate.

Other preferred embodiments of the apparatus of the invention include one or more of the following features:
oscillating means carried by the frame for oscillating the flat plate in the plane of the plate to assist the ripe seeds to roll;
the oscillating means oscillating the flat plate in a direction generally perpendicular to the direction of rolling;
a plurality of flat plates substantially parallel and spaced apart from one another wherein the plates are off set relative to their feed ends;
the feeding means including a first screen positioned above a second screen with smaller openings than the first, wherein both the first and second screens are mounted above the feed end of the flat plate, and wherein the first screen is adapted to receive the components from the hopper, to reciprocate relative to the second screen, and to spread the components over the area of the second screen, and the second screen is adapted to allow the components to pass through by gravity;
an air cleaning system adapted to direct an air blast at the flat plate so as to clean the plate of components which did not roll;
the collection means including a baffle to direct components into separate collection troughs based on time;
the collection means including a gravity slide to gravity feed the components to the baffle;
conveying means, such as augers, in the collection troughs to convey the collected separated components into separate collection bins.

In another broad aspect, the present invention provides a method for separating components of a seed mixture of ripe seeds, unripe seeds, and debris, said apparatus comprising:
holding the mixture of components to be separated;
providing a flat plate with a proximal feed end and a distal discharge end such that the plate declines downwardly from the feed end to the discharge end;
feeding a single layer of components onto the feed end of the flat plate to allow the seeds to roll generally independently;
rolling the components on the flat plate downwardly towards the discharge end wherein the seeds roll at differential rates according to the ripeness of the seeds;
selectively collecting from the discharge end of the plate, at one or more timed intervals, the components of the seed mixture such that ripe seeds are separated as the earliest to reach the discharge end of the plate.

Other preferred embodiments of the method of the invention include one or more of the following features:
oscillating the flat plate in the plane of the plate to assist the ripe seeds to roll;
oscillating the flat plate in a direction generally perpendicular to the direction of rolling;
air blasting the flat plate to clean off any components which did not roll;
conveying separated components to separate collection bins;
the seeds being canola or mustard.

The present invention has been successfully demonstrated with canola and mustard seeds and has application to other oil seeds such as soya beans, as well as peas and other grains on a commercial scale for removing splits, culls, hulls and other debris.

With this method and apparatus, the inventors are able to efficiently separate oil seeds on the basis of ripeness. Prior art machines provide low outputs. For example, to separate 300 bushels of canola per hour requires approximately 10,000 square feet of separating surface. To the inventors' knowledge, no machine has that surface area. To contain such a massive surface into a manageable machine, the method and apparatus of the present invention, preferably provides a plurality of flat plates substantially parallel and spaced apart from one another wherein the plates are staggered relative to their feed ends. This creates a tower of flat plates which would expose tremendous surface area while keeping the overall bulk to a minimum.

One obstacle with having plates in close proximity to each other is how to remove the remaining unripe seeds and debris after the ripe seeds have been separated. The debris includes damaged seeds, dirt, and other foreign bodies like insect bodies. In a preferred embodiment, the method and apparatus provides an air cleaning system which air blasts the flat plate to clean off any components which did not roll.

### BRIEF DESCRIPTION OF THE DRAWINGS

FIG. 1 is a perspective front view of apparatus showing multiple separators attached to a base structure and fan assembly.

FIG. 2 is a perspective front view of apparatus showing multiple separators attached to a base structure without fan assembly and feed cartridges.

FIG. 3 is a perspective rear view of the apparatus showing multiple separators mounted on a base structure, and attached to a fan assembly and a first screen motor.

FIG. 4 is a perspective rear view of the base structure showing collection troughs, and hinged baffle.

FIG. 5 is a plan view of the apparatus showing multiple separators mounted on a base structure, and attached to a fan assembly and a first screen motor.

FIG. 6 is a schematic side elevational view of the method and apparatus of the present invention.

FIG. 7 is a perspective view of the feed cartridge.

### DETAILED DESCRIPTION OF PREFERRED EMBODIMENTS

The apparatus and method of this invention are illustrated in the figures and described in a preferred embodiment with application to separation of canola seeds but not so limited.

The method of the present invention is to feed the given oil seed or grain to be separated onto downwardly declined flat plates so as to allow each seed to roll generally independently down the length of the plate and to be separated by rolling at different rates according to the ripeness of the seeds.

On the basis that ripe seeds roll differentially than unripe seeds, the seeds are collected at timed intervals in separate collection troughs. Ripe seeds are separated as the earliest to roll off the flat plate. Debris and unripe seeds that roll more slowly, remain on the flat plates until they are blown off and are collected into another collection trough. The machine cycles through three timed stages: the feed stage; the run stage; and the clean stage.

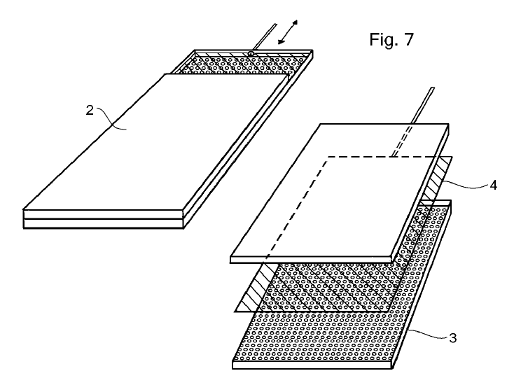

As shown in FIG. 6, pre-cleaned canola seeds are separated by a separator S by first depositing the canola seeds into a hopper 1 mounted on top of the separator S. The canola seeds are then allowed to flow into a feed cartridge 2. As shown in FIG. 7, the bottom of the feed cartridge 2 provides a round-hole screen 3 (hereinafter “second screen”). The holes of the second screen 3 are preferably between 7/64-8/64 of an inch (holes sized to restrict flow of canola seeds, but to allow flow when reciprocating agitation or some form of active disturbance is applied). In a preferred embodiment, a first screen 4, with larger openings than the second screen 3, also housed in the feed cartridge 2, can be placed over the second screen 3. By reciprocating the first screen 4, the canola seeds become agitated and flow. The agitated canola seeds fall through the second screen 3 onto a plurality of flat plates 5 that are off set, substantially parallel and spaced apart from one another. Each flat plate has a proximal feed end 5b (or leading edge) and a distal discharge end 5d. The canola seeds fall onto individual plates loading the leading edge 5b, which is exposed on each successive plate, with a generally single layer of seeds to allow the seeds to roll generally independently.

In a preferred embodiment, these flat plates 5 are 140 glass plates stacked one above another with a distance d₁ between each plate (see FIG. 6). The distance d₁ is sized to allow the passage of seeds down the plurality of flat plates 5 while economizing the space between each plate. In a preferred embodiment d₁ is about 0.25-1.0 inch. For canola seeds d₁ is preferably about 0.25 inches. For larger seeds, like soya or peas, d₁ is preferably 0.5-1.0 inch. In another preferred embodiment, each flat plate is mounted slightly off set the preceding flat plate so that the feed end 5b of each ascending plate protrudes by a distance d₂ (see FIG. 6). The distance d₂ is sized to feed a generally single layer of seeds to feed end 5b of each flat plate. In a preferred embodiment d₂ is about 0.25-1.0 inches. For canola seeds d₂ is preferably about 0.25 inches. For larger seeds, like soya or peas, d₂ is preferably 0.5-1.0 inches. In another preferred embodiment the flat plates 5 are stacked so that the overall stack ascends at a 45 degree slope (see FIG. 6).

In a preferred embodiment the first screen 4 reciprocates relative to the second screen 3, through reciprocating movements of a pivoting arm 25 which has a proximal end attached to the first screen 4 of the feed cartridge 2, and a distal end geared to a first screen motor 29.

As shown in FIG. 6, on contact with a flat plate, ripe canola seeds 8 rolls along the length of the plate away from the proximal feed end 5b and towards the distal discharge end 5d. Each flat plate is preferably declined downwardly about 2-8 degrees from the feed end 5b to the discharge end 5d. In a preferred embodiment, each flat plate is declined downwardly about 4 degrees. The slope of the flat plate can be increased or decreased depending on the level of separation desired. In a preferred embodiment the slope of the flat plate can be changed by placing shims (not shown) under the separator S to tilt the entire separator S at an angle to the horizontal. In another preferred embodiment, hydraulic jacks can be placed under the separator S to incline the separator S and flat plate to a desired angle from the horizontal.

Located proximate the distal discharge ends 5d, is a downwardly declining gravity slide 9. After passing over the flat plate the canola seed 8 falls over discharge end 5d onto the gravity slide 9. At the base of the slide 9 is a hinged baffle 10, which directs the seeds 8 into one or more collection troughs 11. From the collection troughs 11, the canola is then conveyed by augers 27 to its appropriate collection bin. The run stage should be sufficiently long to maximize the different rolling resistance of the two types of seeds.

In a preferred embodiment the gravity slide 9 is made of a mesh material with openings sized to retain canola seeds and debris. This aids in air cleaning the slide.

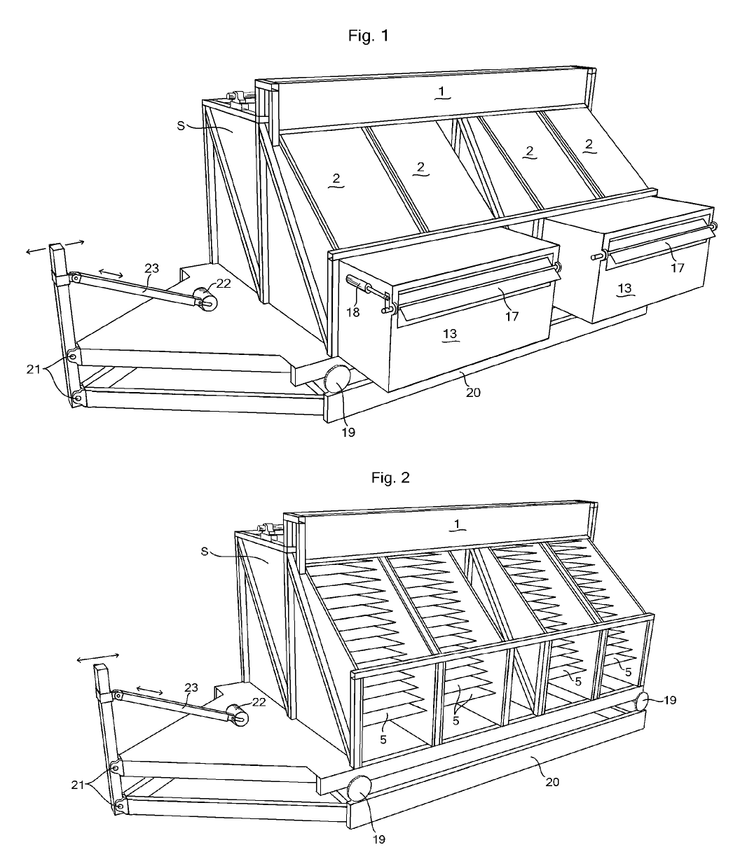

As shown in FIGS. 1, 5, and 6, connected to the separator S is a fan 13 adapted with fan baffles 17 that are hydraulically controlled to open and close through a hydraulic cylinder 18. When the fan baffles 17 are in the open position, a blast of air is generated by the fan 13 through the front of the separator S and directed towards the plurality of flat plates 5. This blast of air blows out unripe canola seeds and debris which have not rolled out and still remain on the plurality of flat plates at the end of the run cycle. The unripe seeds and debris are trapped against the gravity slide 9 by this blast of air until the end of the clean stage, after which the blast of air is ceased by closing the fan baffles 17 and the unripe seeds and debris are directed down the slide 9 to the hinged baffle 10 which directs into one or more collection troughs 12. From the collection troughs 12, the canola is then conveyed by augers 28 into its appropriate collection bin. It usually requires 2-5 seconds to clear the plurality of flat plates 5 with the blast of air. Immediately after closing the fan baffles 17 and ceasing the blast of air, the separator S returns to the feed stage.

In a preferred embodiment the hinged baffle 10 is controlled by a hydraulic cylinder 26 that pivots the hinged baffle 10 to direct the seeds 8 into specific collection troughs 11 or 12 (see FIG. 6).

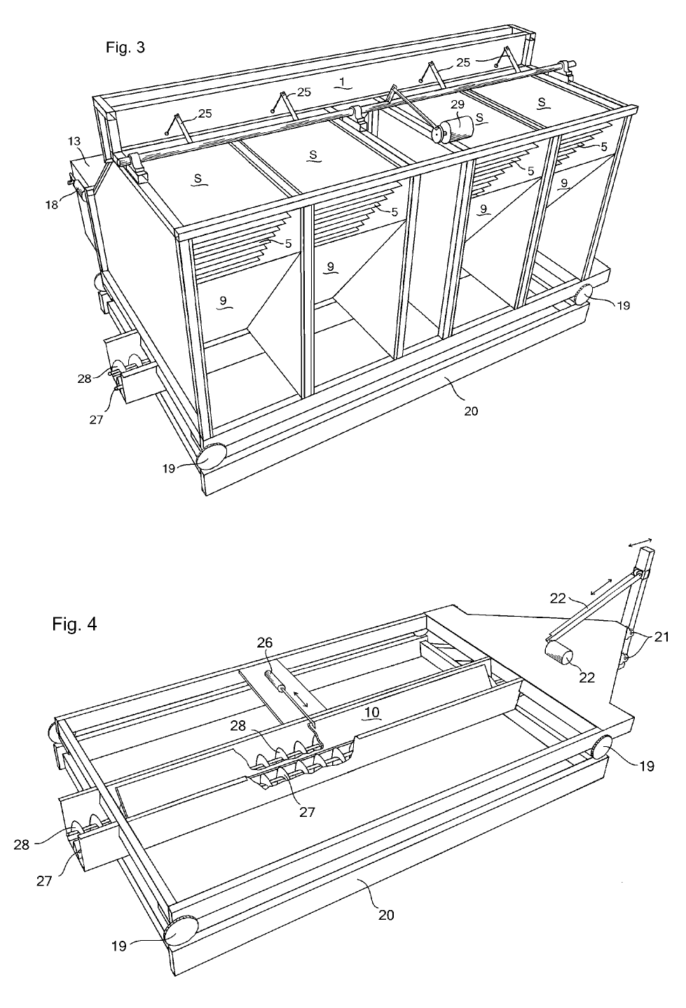

As shown in FIGS. 1 through 6, the separator S is mounted on wheels 19 positioned on a base 20. The separator S and the base 20 are articulated to a pivoting oscillating arm 23 with pivots 21. The oscillating arm 23 is in turn connected to an oscillation motor 22 such that revolutions of the oscillation motor 22 reciprocate the pivoting oscillating arm 23. As the oscillating arm 23 reciprocates the separator S rolls on wheels 19 in a back and forth motion relative to the base 20 in a generally perpendicular direction to the direction of seed rolling along the flat plates 5. Oscillation of sufficient frequency and force assists rolling of ripe seeds while keeping the unripe seeds in place. The extent of the oscillation is controlled by raising or lowering the height of the oscillating arm.

In a preferred embodiment the air blast is created by a high volume fan. At the end of the run stage a signal is sent to open the fan baffles 17, thus allowing the blast of air to be directed into and through the separator S.

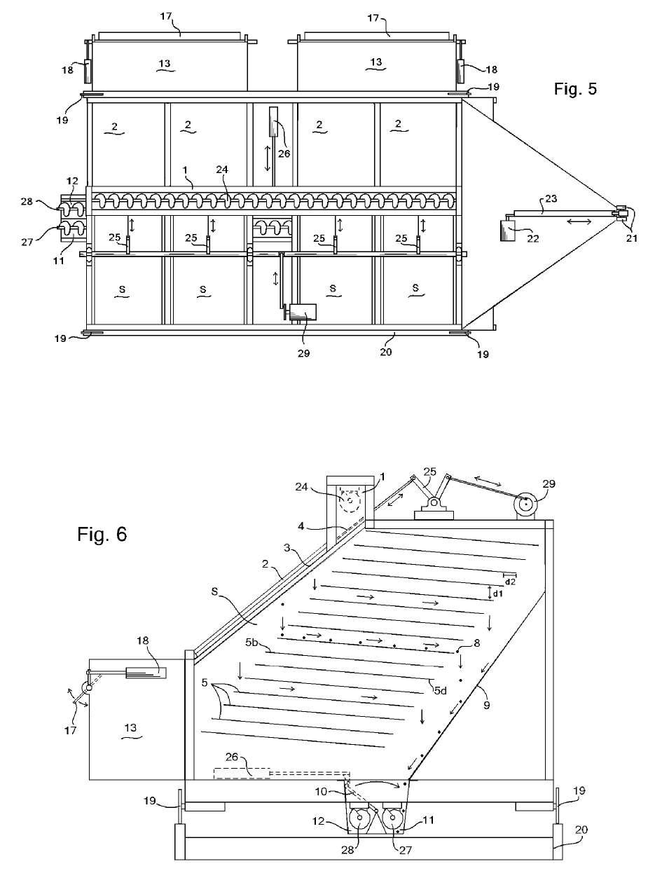

As shown in FIGS. 3 and 5, the invention can be adapted to accommodate a plurality of separators S. In such an embodiment a cross auger 24 mounted above the separators S inside the hopper 1 to distribute canola seeds to multiple feed cartridges 2.

To achieve a higher degree of separation, that is to further increase the grade of the canola, the ripe canola seeds collected after a run stage can be re-fed into the hopper 1 to be processed through the separator S more than once. In so doing, a higher degree of separation can be achieved with each successive pass. In the same manner, the unripe canola seeds collected after the run stage can be re-fed and processed through the separator S more than once in order to draw out any ripe canola seeds that might have been trapped on the plurality of flat plates 5 during the clean stage.

Changing the slope of the plurality of flat plates 5 effects both the green seed count and the yield of canola. Increasing slope will increase the yield of canola, but will also increase the green seed count. Decreasing the slope tends to reduce the green seed count of the canola, while sacrificing yield. The ideal slope depends on the grade of canola being fed into the separator S, and the grade/yield of canola desired. For example, if the incoming canola is 10% green then you could collect a No. 1 grade canola sample with a 6 degree downward decline. If the green count is higher, then to collect a No. 1 grade canola sample a downward decline of 2-3 degrees would be suggested. In a preferred embodiment the present invention operates with the plurality of flat plates 5 at a downward decline of 2-8 degrees from the proximal feed end 5b to the distal discharge end 5d.

Changing the force and frequency of oscillation has much the same effect as changing the slope. Increasing force leads to increased yield, and vice versa. In a preferred embodiment the present invention operates with a stroke length of 0.25-0.75 of an inch and a frequency of 150-200 rpm.

In an alternative embodiment a third higher grade trough can be added to the base of the machine to improve the refinement capability. In the course of testing, the inventors discovered that there were less green seeds collected early in the run stage than were collected later in the run stage. By incorporating a third trough to separate this early portion from later portions, a higher grade supply of ripe canola seeds can be collected in addition to the other grades.

In addition to the embodiments listed above there are possible variations to the apparatus that can be made without departing in any way from the present invention. One such variation is the insertion of vertical or almost vertical tabs along the leading edge 5b of each flat plate so as to minimize overflow and help guide the canola seeds onto its appropriate flat plate. Another variation is adding a hinged baffle to fan exit to direct air in a sweeping pattern across the plurality of flat plates 5 during the clean stage. In yet another variation a vibrator can be used in place of an agitator. In another variation a vibrator is connected to the separator S to vibrate the flat plate to assist the ripe seeds to roll. In another variation the second screen 3 of the feed cartridge 2 is adapted with openings that open and close to selectively allow seeds to fall through the second screen 3 onto the plurality of flat plates 5

All publications mentioned in this specification are indicative of the level of skill in the art of this invention. All publications are herein incorporated by reference to the same extent as if each publication was specifically and individually indicated to be incorporated by reference. The terms and expressions used are, unless otherwise defined herein, used as terms of description and not limitation. There is no intention, in using such terms and expressions, of excluding equivalents of the features illustrated and described.

1. An apparatus for separating components of a seed mixture, said apparatus comprising:
(a) a frame;
(b) a flat plate connected to the frame with a proximal feed end and a distal discharge end, wherein the flat plate is positioned to decline downwardly from the feed end to the discharge end so as to allow the components to be separated by rolling at different rates according to the roundness of the seeds;
(c) feeding means attached to the frame for feeding the components onto the feed end of the flat plate as a generally single layer of seeds to allow the seeds to roll generally independently;
(d) oscillating means attached to the frame for oscillating the flat plate in the plane of the plate to assist the seeds to roll.
(e) collection means at the discharge end of the flat plate adapted for selectively collecting, at one or more timed intervals, the components of the seed mixture such that round seeds are separated as the earliest to reach the discharge end of the plate.

2. The apparatus of claim 1, further comprising a plurality of flat plates substantially parallel and spaced apart from one another wherein the plates are staggered relative to their feed ends.

3. The apparatus of claim 2, wherein the oscillating means oscillates the flat plate in a direction generally perpendicular to the direction of rolling.

4. The apparatus of claim 3, further comprising an air cleaning system adapted to direct an air blast at the flat plate so as to clean the plate of components which did not roll.

5. The apparatus of claim 4, wherein the collection means includes a baffle to direct components into separate collection troughs based on time.

6. The apparatus of claim 5, wherein the collection means includes a gravity slide to gravity feed the components to the baffle.

7. The apparatus of claim 6, further comprising conveying means in the collection troughs to move the collected separated components into separate collection bins.

8. The apparatus of claim 7, wherein the air cleaning system comprises a fan adapted with fan baffles wherein the fan baffles gate the flow of air wherein the flow of air allows the fan to generate the air blast.

9. The apparatus of claim 8, wherein the conveying means are augers.

10. The apparatus of claim 8, wherein the means for oscillating the flat plate includes a pivoting arm articulated with the frame and a stationary base, wherein reciprocating the pivoting arm oscillates the flat plate relative to the stationary base.

11. The apparatus of claim 8, wherein the feeding means includes a first screen positioned above a second screen with smaller openings than the first, wherein both the first and second screens are mounted above the feed end of the flat plate, and wherein the first screen is adapted to receive the components from the hopper, to reciprocate relative to the second screen, and to spread the components over the area of the second screen, and the second screen is adapted to allow the components to pass through by gravity.

12. The apparatus of claim 8, wherein the feeding means includes a pivoting arm geared to a motor and attached to the first screen for reciprocating the first screen relative to the second screen.

13. The apparatus of claim 8, further comprising a return trough carried by the frame and adapted beneath the plurality of flat plates to collect components that pass through the second screen but are not fed on the feed end of the flat plates.

14. A method for separating components of a seed mixture of ripe seeds, unripe seeds, and debris, said apparatus comprising:
(a) providing a flat plate with a proximal feed end and a distal discharge end such that the plate declines downwardly from the feed end to the discharge end;
(b) feeding a single layer of components onto the feed end of the flat plate to allow the seeds to roll generally independently;
(c) rolling the components on the flat plate downwardly towards the discharge end wherein the seeds roll at differential rates according to the ripeness of the seeds;
(d) selectively collecting from the discharge end of the plate, at one or more timed intervals, the components of the seed mixture such that ripe seeds are separated as the earliest to reach the discharge end of the plate.

15. The method of claim 14, further comprising oscillating the flat plate in the plane of the plate to assist the ripe seeds to roll.

16. The method of claim 15, further comprising oscillating the flat plate in a direction generally perpendicular to the direction of rolling.

17. The method of claim 16, further comprising air blasting the flat plate to clean off any components which did not roll.

18. The method of claim 17, further comprising conveying separated components to separate collection bins.

19. The method of claim 14, wherein the seeds are canola.

20. The method of claim 14, wherein the seeds are mustard.
